SEQUENCE LISTING (61 sequences)

SEQ ID NO: 1 - protein, 118 aa, Artificial Sequence
  synthetic anti-EphA3 antibody heavy chain V-region (V-H)
QVQLVQSGAEVKKPGTSVKVSCKASGYTFTGYWMNWVRQASGQGLEWMGDIYPGSGNTNYDEKFQGRVTMTRDTSISTAYMELSRLRSDDTAVYYCARSGVYEDFDSWGQGTTVTVSS

SEQ ID NO: 2 - protein, 118 aa, Artificial Sequence
  synthetic anti-EphA3 antibody heavy chain V-region (V-H)
QVQLVQSGAEVKKPGASVKVSCKASGYTFTGYWMNWVRQAPGQGLEWMGDIYPGSGNTNYDEKFQGRVTMTRDTSISTAYMELSRLRSDDTAVYYCARSGYYEEFDSWGQGTTVTVSS

SEQ ID NO: 3 - protein, 118 aa, Artificial Sequence
  synthetic anti-EphA3 antibody heavy chain V-region (V-H)
QVQLVQSGAEVKKPGTSVKVSCKASGYTFTGYWMNWVRQASGQGLEWMGDIYPGSGNTNYDEKFQGRVTMTRDTSISTAYMELSRLRSDDTAVYYCARSGYYEEFDSWGQGTTVTVSS

SEQ ID NO: 4 - protein, 118 aa, Artificial Sequence
  synthetic anti-EphA3 antibody heavy chain V-region (V-H)
QVQLVQSGAELKKPGASVKVSCKTSGYTFTGYWMNWVRQAPGQGLEWMGDIYPGSGNTNYDEKFQGRVTMTRDTSISTAYMELSRLRSDDTAVYYCARSGYYEEFDSWGQGTTVTVSS

SEQ ID NO: 5 - protein, 118 aa, Artificial Sequence
  synthetic anti-EphA3 antibody heavy chain V-region (V-H)
QVQLVQSGAEVKKPGASVKVSCKASGYTFTGYWMNWVRQAPGQGLEWMGDIYPGSGNTNYDEKFQGRVTMTRDTSISTAYMELSRLRSDDTAVYYCARGGYYEDFDSWGQGTTVTVSS

SEQ ID NO: 6 - protein, 118 aa, Artificial Sequence
  synthetic anti-EphA3 antibody heavy chain V-region (V-H)
EVQLVQSGAEVKKPGESLRISCKGSGYTFTTYWISWVRQMPGQGLEWMGDIYPGSGNTNYAQKFQGRVTITADKSTSTAYMGLSSLRSEDTAVYYCARSGYYEDFDSWGQGTTVTVSS

SEQ ID NO: 7 - protein, 118 aa, Artificial Sequence
  synthetic anti-EphA3 antibody heavy chain V-region (V-H)
EVQLVQSGAEVKKPGESLRISCKGSGYTFTTYWISWVRQMPGQGLEWMGDIYPGSGNTNYAQEFRGRVTITADESTSTAYVELSSLRSEDTAVYYCARSGYYEDFDSWGQGTTVTVSS

SEQ ID NO: 8 - protein, 118 aa, Artificial Sequence
  synthetic anti-EphA3 antibody heavy chain V-region (V-H)
EVQLVQSGAEVKKPGESLRISCKGSGYTFTTYWISWVRQMPGQGLEWMGDIYPGSGNTNYAQKFLGRVTITADESTSTAYMELSSLRYDDTAVYYCARSGYYEDFDSWGQGTTVTVSS

SEQ ID NO: 9 - protein, 118 aa, Artificial Sequence
  synthetic anti-EphA3 antibody heavy chain V-region (V-H)
EVKLEESGAELVKPGSSVKLSCKASGYNFTSYWINWVRLRPGQGLEWIGDIYPGSGNTNYDEKFKRKATLTVDTSSSTAYMQLSSLASEDTAVYYCARSGYYEDFDVWGQGTTVTVSS

SEQ ID NO: 10 - protein, 118 aa, Artificial Sequence
  synthetic anti-EphA3 antibody heavy chain V-region (V-H)
EVKLEESGAELVKPGSSVKLSCKASGYNFTSYWINWVRLRPGQGLEWIGDIYPGSGNTNYDEKFKRKATLTVDTSSSTAYMQLSSLASEDTAVYYCARSGYYEDFDIWGQGTMVTVSS

SEQ ID NO: 11 - protein, 107 aa, Artificial Sequence
  synthetic anti-EphA3 antibody light chain V-region (V-L)
DIQMTQSPSFLSASVGDRVTITCRASQGIISYLAWYQQKPEKAPKRLIYAASSLQSGVPSRFSGSGSGTEFTLTISSLQPEDFATYYCVQYANYPYTFGQGTKLEIK

SEQ ID NO: 12 - protein, 107 aa, Artificial Sequence
  synthetic anti-EphA3 antibody light chain V-region (V-L)
DIQMTQSPSFLSASVGDRVTITCRASQGIISYLAWYQQKPEKAPKRLIYAASSLQSGVPSRFSGSGSGTEFTLTISSLQPEDFATYYCVQYSNYPYTFGQGTKLEIK

SEQ ID NO: 13 - protein, 107 aa, Artificial Sequence
  synthetic anti-EphA3 antibody light chain V-region (V-L)
DIQMTQSPSFLSASVGDRVTITCRASQGIISYLAWYQQKPEKAPKRLIYAASSLQSGVPSRFSGSGSGTEFTLTISSLQPEDFATYYCGQYANYPYTFGQGTKLEIK

SEQ ID NO: 14 - protein, 107 aa, Artificial Sequence; identical to SEQ ID NO: 11
  synthetic anti-EphA3 antibody light chain V-region (V-L)

SEQ ID NO: 15 - protein, 107 aa, Artificial Sequence
  synthetic anti-EphA3 antibody light chain V-region (V-L)
DIQLTQSPSFLSASVGDRVTITCRASQGIISYLAWYQQKPEKAPKRLIYGASSLQSGVPSRFSGSGSGTEFTLTISSLQPEDFATYYCVQYANYPYTFGQGTKLEIK

SEQ ID NO: 16 - protein, 107 aa, Artificial Sequence
  synthetic anti-EphA3 antibody light chain V-region (V-L)
DIQMTQSPSSLSASLGDRVTITCQASQDISTYLNWIQQKPGKAPKRLIYAASSLQSGVPSRFSGSGSGTEFTLTISSLQPEDFATYYCVQYANYPYTFGQGTKLEIK

SEQ ID NO: 17 - protein, 107 aa, Artificial Sequence
  synthetic anti-EphA3 antibody light chain V-region (V-L)
DIQMTQSPSSLSASLGDRVTITCQASQDISTYLNWIQQKPGKAPKRLIYAASSLQRGVPSRFSGSGSGTEFTLTISSLQPEDFATYYCVQYANYPYTFGQGTKLEIK

SEQ ID NO: 18 - protein, 107 aa, Artificial Sequence
  synthetic anti-EphA3 antibody light chain V-region (V-L)
DIQMTQSPSSLSASVGDRVTITCRASQSISSYLAWYQQKPGKAPKRLIYAASSLQRGVPSRFSGSGSGTEFTLTISSLQPEDFATYYCVQYANYPYTFGQGTKLEIK

SEQ ID NO: 19 - protein, 107 aa, Artificial Sequence
  synthetic anti-EphA3 antibody light chain V-region (V-L)
DIQMTQSPSFLSASVGDRVTITCRASQGIISYLAWYQQKPEKAPKRLIYAASSLQSGVPSRFSGSGSGTEFTLTISSLQPEDFATYYCAQYANYPYTFGQGTKLEIK

SEQ ID NO: 20 - protein, 107 aa, Artificial Sequence
  synthetic anti-EphA3 antibody light chain V-region (V-L)
DIQMTQSPSFLSASVGDRVTITCRASQGIISYLAWYQQKPEKAPKRLIYAASSLQSGVPSRFSGSGSGTEFTLTISSLQPEDFATYYCVQYAKYPYTFGQGTKLEIK

SEQ ID NO: 21 - protein, 107 aa, Artificial Sequence
  synthetic anti-EphA3 antibody light chain V-region (V-L)
DIQMTQSPSFLSASVGDRVTITCRASQGIISYLAWYQQKPEKAPKRLIYAASSLQSGVPSRFSGSGSGTEFTLTISSLQPEDFATYYCVGYANYPYTFGQGTKLEIK

SEQ ID NO: 22 - protein, 107 aa, Artificial Sequence
  synthetic anti-EphA3 antibody light chain V-region (V-L)
DIQMTQSPSFLSASVGDRVTITCRASQGIISYLAWYQQKPEKAPKRLIYAASSLQSGVPSRFSGSGSGTEFTLTISSLQPEDFATYYCVRYANYPYTFGQGTKLEIK

SEQ ID NO: 23 - protein, 107 aa, Artificial Sequence
  synthetic anti-EphA3 antibody light chain V-region (V-L)
DIQMTQSPSFLSASVGDRVTITCRASQGIISYLAWYQQKPEKAPKRLIYAASSLQSGVPSRFSGSGSGTEFTLTISSLQPEDFATYYCVQYLNYPYTFGQGTKLEIK

SEQ ID NO: 24 - protein, 9 aa, Artificial Sequence
  synthetic anti-EphA3 antibody heavy chain V-region (V-H) CDR3
XGXYEXFDX

SEQ ID NO: 25 - protein, 9 aa, Artificial Sequence
  synthetic anti-EphA3 antibody heavy chain V-region (V-H) CDR3
GGYYEDFDS

SEQ ID NO: 26 - protein, 9 aa, Artificial Sequence
  synthetic anti-EphA3 antibody heavy chain V-region (V-H) CDR3
SGYYEEFDS

SEQ ID NO: 27 - protein, 9 aa, Artificial Sequence
  synthetic anti-EphA3 antibody heavy chain V-region (V-H) CDR3
SGVYEDFDS

SEQ ID NO: 28 - protein, 9 aa, Artificial Sequence
  synthetic anti-EphA3 antibody heavy chain V-region (V-H) CDR3
SGYYEDFDV

SEQ ID NO: 29 - protein, 9 aa, Artificial Sequence
  synthetic anti-EphA3 antibody heavy chain V-region (V-H) CDR3
SGYYEDFDI

SEQ ID NO: 30 - protein, 10 aa, Artificial Sequence
  synthetic anti-EphA3 antibody heavy chain FR4
WGQGTTVTVS

SEQ ID NO: 31 - protein, 5 aa, Artificial Sequence
  synthetic anti-EphA3 antibody heavy chain V-region (V-H) CDR1, anti-EphA3 antibody FA225-C10 CDRH1
GYWMN

SEQ ID NO: 32 - protein, 5 aa, Artificial Sequence
  synthetic anti-EphA3 antibody heavy chain V-region (V-H) CDR1, anti-EphA3 antibody FA3AM-H12A and K3D CDRH1
TYWIS

SEQ ID NO: 33 - protein, 5 aa, Artificial Sequence
  synthetic anti-EphA3 antibody heavy chain V-region (V-H) CDR1, anti-EphA3 antibody IIIA4 CDRH1
SYWIN

SEQ ID NO: 34 - protein, 17 aa, Artificial Sequence
  synthetic anti-EphA3 antibody heavy chain V-region (V-H) CDR2, anti-EphA3 antibody FA3AM-H12A and FA225-C10 CDRH2
DIYPGSGNTNYDEKFQG

SEQ ID NO: 35 - protein, 17 aa, Artificial Sequence
  synthetic anti-EphA3 antibody heavy chain V-region (V-H) CDR2
DIYPGSGNTNYAQKFQG

SEQ ID NO: 36 - protein, 17 aa, Artificial Sequence
  synthetic anti-EphA3 antibody heavy chain V-region (V-H) CDR2, anti-EphA3 antibody K3D CDRH2
DIYPGSGNTNYAQEFRG

SEQ ID NO: 37 - protein, 17 aa, Artificial Sequence
  synthetic anti-EphA3 antibody heavy chain V-region (V-H) CDR2
DIYPGSGNTNYAQKFLG

SEQ ID NO: 38 - protein, 17 aa, Artificial Sequence
  synthetic anti-EphA3 antibody heavy chain V-region (V-H) CDR2
DIYPGSGNTNYDEKFEG

SEQ ID NO: 39 - protein, 17 aa, Artificial Sequence
  synthetic anti-EphA3 antibody heavy chain V-region (V-H) CDR2, anti-EphA3 antibody IIIA4 CDRH2
DIYPGSGNTNYDEKFKR

SEQ ID NO: 40 - protein, 17 aa, Artificial Sequence
  synthetic anti-EphA3 antibody heavy chain V-region (V-H) CDR2
DIYPGSGNTNYAQXFXG

SEQ ID NO: 41 - protein, 9 aa, Artificial Sequence
  synthetic anti-EphA3 antibody light chain V-region (V-L) CDR3 binding specificity determinant (BSD)
XXYXXYPYT

SEQ ID NO: 42 - protein, 9 aa, Artificial Sequence
  synthetic anti-EphA3 antibody light chain V-region (V-L) CDR3, anti-EphA3 antibody FA225-C10 CDRL3
GQYANYPYT

SEQ ID NO: 43 - protein, 9 aa, Artificial Sequence
  synthetic anti-EphA3 antibody light chain V-region (V-L) CDR3
VQYAKYPYT

SEQ ID NO: 44 - protein, 9 aa, Artificial Sequence
  synthetic anti-EphA3 antibody light chain V-region (V-L) CDR3
AQYANYPYT

SEQ ID NO: 45 - protein, 9 aa, Artificial Sequence
  synthetic anti-EphA3 antibody light chain V-region (V-L) CDR3
VQYSNYPYT

SEQ ID NO: 46 - protein, 9 aa, Artificial Sequence
  synthetic anti-EphA3 antibody light chain V-region (V-L) CDR3, anti-EphA3 antibody IIIA4 and FA3AM-H12A CDRL3
VQYANYPYT

SEQ ID NO: 47 - protein, 9 aa, Artificial Sequence
  synthetic anti-EphA3 antibody light chain V-region (V-L) CDR3
VGYANYPYT

SEQ ID NO: 48 - protein, 9 aa, Artificial Sequence
  synthetic anti-EphA3 antibody light chain V-region (V-L) CDR3
VRYANYPYT

SEQ ID NO: 49 - protein, 9 aa, Artificial Sequence
  synthetic anti-EphA3 antibody light chain V-region (V-L) CDR3, anti-EphA3 antibody FA225-C10 CDRL3
VQYLNYPYT

SEQ ID NO: 50 - protein, 9 aa, Artificial Sequence
  synthetic human germ-line Jkappa2 J segment, anti-EphA3 antibody FR4
FGQGKLEIK

SEQ ID NO: 51 - protein, 11 aa, Artificial Sequence
  synthetic anti-EphA3 antibody light chain V-region (V-L) CDR1, anti-EphA3 antibody FA3AM-H12A, K3D and FA225-C10 CDRL1
RASQGIISYLA

SEQ ID NO: 52 - protein, 11 aa, Artificial Sequence
  synthetic anti-EphA3 antibody light chain V-region (V-L) CDR1
QASQDISTYLN

SEQ ID NO: 53 - protein, 11 aa, Artificial Sequence
  synthetic anti-EphA3 antibody light chain V-region (V-L) CDR1, anti-EphA3 antibody IIIA4 CDRL1
RASQEISGYLG

SEQ ID NO: 54 - protein, 11 aa, Artificial Sequence
  synthetic anti-EphA3 antibody light chain V-region (V-L) CDR1
RASQSISSYLA

SEQ ID NO: 55 - protein, 7 aa, Artificial Sequence
  synthetic anti-EphA3 antibody light chain V-region (V-L) CDR2, anti-EphA3 antibody FA3AM-H12A, K3D and FA225-C10 CDRL2
AASSLQS

SEQ ID NO: 56 - protein, 7 aa, Artificial Sequence
  synthetic anti-EphA3 antibody light chain V-region (V-L) CDR2
GASSLQS

SEQ ID NO: 57 - protein, 7 aa, Artificial Sequence
  synthetic anti-EphA3 antibody light chain V-region (V-L) CDR2
AASSLQR

SEQ ID NO: 58 - protein, 7 aa, Artificial Sequence
  synthetic anti-EphA3 antibody light chain V-region (V-L) CDR2, anti-EphA3 antibody IIIA4 CDRL2
AASTLDS

SEQ ID NO: 59 - protein, 9 aa, Artificial Sequence
  synthetic anti-EphA3 antibody heavy chain V-region (V-H) CDR3
SGYYEXFDS

SEQ ID NO: 60 - protein, 9 aa, Artificial Sequence
  synthetic anti-EphA3 antibody IIIA4 heavy chain V-region (V-H) CDR3 (CDRH3)
SGYYEDFDS

SEQ ID NO: 61 - protein, 9 aa, Artificial Sequence
  synthetic anti-EphA3 antibody K3D light chain V-region (V-L) CDR3 (CDRL3)
VQYMNYPYT

(19) United States
(12) Patent Application Publication
(10) Pub. No.: US 2011/0243934 A1
(43) Pub. Date: Oct. 6, 2011

(54) EPHA3 ANTIBODIES FOR THE TREATMENT OF MULTIPLE MYELOMA

(75) Inventors: Varghese Palath, South San Francisco, CA (US); Christopher R. Bebbington, South San Francisco, CA (US)

(73) Assignee: KaloBios Pharmaceuticals Inc., South San Francisco, CA (US)

(21) Appl. No.: 13/078,747

(22) Filed: Apr. 1, 2011

Related U.S. Application Data
(60) Provisional application No. 61/320,230, filed on Apr. 1, 2010.

(51) Int. Cl. A61K 39/395; A61P 35/00; C12Q 1/02; C12Q 1/68

(52) U.S. Cl. 424/133.1; 424/174.1; 435/29; 435/6.1

(57) ABSTRACT

The invention provides methods and compositions comprising anti-EphA3 antibodies for the treatment of multiple myeloma.

### CROSS-REFERENCE TO RELATED APPLICATIONS

This application claims benefit of U.S. application 61/320,230 filed Apr. 1, 2010, which application is herein incorporated by reference for all purposes.

### BACKGROUND OF THE INVENTION

Eph receptor tyrosine kinases (Ephs) belong to a large group of receptor tyrosine kinases (RTKs), kinases that phosphorylate proteins on tyrosine residues. Ephs and their membrane bound ephrin ligands (ephrins) control cell positioning and tissue organization (Poliakov, A., et al., Dev Cell 7:465-80 (2004)). In contrast to other receptor tyrosine kinases, Eph receptor activation does not only require ligand binding and dimerization, but also involves preformed ligand oligomers. Thus, tyrosine phosphorylation of Eph receptors requires presentation of ephrin ligands in their clustered or membrane-attached forms (Davis et al., Science 266:816-819 (1994)). Functional and biochemical Eph responses occur at higher ligand oligomerization states (Stein et al., Genes Dev 12:667-678 (1998)).

Among other patterning functions, various Ephs and ephrins have been shown to play a role in vascular development. The de-regulated re-emergence of some ephrins and their receptors in adults also has been observed to contribute to tumor invasion, metastasis and neo-angiogenesis. For example, dominant-negative, soluble EphA2 or A3 proteins exhibit effects on ephrin-induced endothelial cell function in vitro, and tumor angiogenesis and progression in vivo (Nakamoto, et al., Microsc Res Tech 59:58-67 (2002); Brantley-Sieders, et al., Curr Pharm Des 10:3431-42 (2004); Brantley, et al. Oncogene 21:7011-26 (2002); Cheng, et al. Neoplasia 5:445-56 (2003). Dobrzanski, et al. Cancer Res 64:910-9 (2004)). Furthermore, Eph family members have been found to be over-expressed on tumor cells from a wide variety of human solid tumors (Brantley-Sieders, et al., Curr Pharm Des 10:3431-42 (2004); Marme, D. Ann Hematol 81 Suppl 2:S66 (2002); Booth, C. et al., Nat Med 8:1360-1 (2002)).

Approximately 15,000 new cases of multiple myeloma are diagnosed each year in the United States. Multiple myeloma is a plasma cell neoplasm characterized by malignant proliferation of plasma cells derived from a single clone. Its etiology is unknown, though it has been associated with exposure to large levels of radiation and certain types of work environments (farming, wood- and leather-working) and is rarely found in persons under the age of forty. The disease generally presents as movement-exacerbated bone pain. Bone lesions, which may result in fracture, are a consequence of proliferation of tumor cells in the bone marrow and activation of osteoclasts, which destroy the bone. The breakdown of bone also leads to increased blood calcium levels. Other common symptoms of the disease include renal failure and anemia. Urinary excretion of immunoglobulin light chains, so-called “Bence Jones” proteins is a consequence of overproduction of the monoclonal immunoglobulin, the “M” protein, which is most typically an IgG, but may also be IgA or IgM, and rarely IgD or IgE. In other cases, a single species of heavy or light chain may be secreted. Elevated serum levels of the “M” protein may be measured by serum protein electrophoresis; however, such elevated levels of M protein (and concomitant urinary secretion of Bence Jones proteins) may be absent in about 1% of “non-secreting” patients. Various chromosomal translocations have also been observed in multiple myeloma patients; a frequent translocation between the immunoglobulin heavy chain gene 14q32 and one of various oncogenes (such as 11q13) results in dysregulation of the oncogene and proliferation of a plasma cell clone.

While there are various treatments available for multiple myeloma (e.g, proteasome inhibitors, thalidomide and thalidomide derivatives), at present none is considered curative. Recently, high dose, immunoablative chemotherapy followed by hematopoietic stem cell transplantation has been used for patients who are sufficiently healthy to withstand the treatment. Autologous transplantation (patient's own stem cells) is most commonly used, but is considered life-prolonging, not curative; allogeneic (healthy donor's stem cells) may be curative, but is not widely available, due to the need for a matched donor. Subsequent treatment of these patients, as well as patients not healthy enough to tolerate transplantation include use of melphalan and prednisone, or more recently, these drugs in conjunction with the proteasome inhibitor bortezomib. Other regimens include thalidomide or lenalidomide (a thalidomide derivative) in conjunction with low dose dexamethasone.

Thalidomide's efficacy in multiple myeloma has been attributed to both anti-angiogenic and pro-apoptotic properties of the molecule. In studies on the U266 multiple myeloma cell line, Liu et al. (The Hematology J. 5: 247-254, 2004) showed up- and down-regulation of a number of genes known to be involved in these processes. EphA3 was reported among ten of genes not necessarily involved in either of these processes, but most significantly up-regulated by s-thalidomide in these studies; however, no evidence of change in EphA3 protein expression was provided, nor was there any indication that activation of EphA3 was involved in the apoptotic process. More recently, it has been reported that the tyrosine kinase inhibitor dasatinib, either alone or in combination with other anti-neoplastic chemotherapeutic agents or an HMG-CoA inhibitor, may be useful in treating multiple myeloma, based on in vitro data showing activity of dasatinib in killing primary multiple myeloma cells (Mitsiades, et al. WO2007059078).

The present invention is based, in part, on the discovery that EphA3 is expressed on the cell surface of primary multiple myeloma cells obtained from patients and further, that binding of an anti-EphA3 antibody to these cells is effective in inducing apoptosis and antibody-dependent cell-mediated cytotoxicity (ADCC).

### BRIEF SUMMARY OF THE INVENTION

The invention is based, in part, on the discovery that multiple myeloma cells obtained from a patient who has multiple myeloma express EphA3. Thus, in one aspect, the invention provides a method of killing multiple myeloma cells, the method comprising contacting the cells with an anti-EphA3 antibody. In some embodiments, the anti-EphA3 antibody is contacted with the cells in vivo. In some embodiments, the multiple myeloma cells are characterized by other antigenic characteristics, such as lack of CD138 antigen and presence of CD19 antigen and/or CD27 antigen (CD138⁻CD19⁺; CD138⁻CD27⁺; CD138⁻CD27⁺CD19⁺). In some embodiments, the anti-EphA3 antibody activates EphA3. In some embodiments, the anti-EphA3 antibody kills the target cells by apoptosis. In some embodiments, the anti-EphA3 antibody kills the target cells by antibody-dependent cell-mediated cytotoxicity (ADCC). In some embodiments, the cell killing is attributable to both apoptosis and ADCC. In some embodiments, the anti-EphA3 antibody is a recombinant or chimeric antibody. In some embodiments, the anti-EphA3 antibody is a human antibody. The anti-EphA3 antibody may be a polyclonal antibody or a monoclonal antibody. In some embodiments, the anti-EphA3 antibody is a multivalent antibody that comprises a Fab, a Fab′, or an Fv. In some embodiments, the antibody is a F(ab′)₂.

In some embodiments, an anti-EphA3 antibody for use in the methods of the invention, e.g., for killing multiple myeloma cells, competes for EphA3 binding with mAb IIIA4. In some embodiments the anti-EphA3 antibody comprises the V_{H} and V_{L} regions of mAb IIIA4. In some, embodiments, the anti EphA3 antibody comprises the V_{H} and V_{L} region CDR1, CDR2 and CDR3 of mAb IIIA4. In some embodiments, the antibody comprises the V_{H} region CDR3 and V_{L} region CDR3 of mAb IIIA4. In some embodiments, an anti-EphA3 antibody for use in the invention comprises a V_{H} region that comprises a CDR3 comprising the amino acid sequence X₁GX₂YEX₃FDX₄, wherein X₁ is S or G, X₂ is Y or V, X₃ is E or D, and X₄ is S, V, or I. In some embodiments, the antibody has a CDR3 that comprises the amino acid sequence GGYYEDFDS, SGYYEEFDS, SGVYEDFDS, SGYYEDFDV, or SGYYEDFDI. In some embodiments, the antibody has a J segment that comprises at least 80% identity, typically at 85%, or at least 90% identity, to a human germline J segment amino acid sequence; or that differs from a human germline J segment at no more than two positions; and a V-segment that comprises at least 80% identity, typically at least 85% identity, and preferably 90% identity, or greater, to a human germ line V-segment amino acid sequence, or at least 80% identity, typically at least 85% identity, and preferably 90% identity, or greater to the framework region (i.e, the V-segment sequence excluding the CDRs). In some embodiment, the J segment comprises at least 90% identity to human JH6 amino acid sequence, and the V-segment comprises at least 90% identity to a human VH1 1-02 amino acid sequence. In some embodiments, the antibody has an FR4 that comprises WGQGTTVTVS, or an FR4 that differs no more than one amino acid from WGQGTTVTVS. In some embodiments, the antibody comprises a V_{H} CDR1, or a V_{H} CDR2, or both a V_{H} CDR1 and a V_{H} CDR2, as shown in a V_{H} region set forth in FIG. 4. For example, in some embodiments, an anti-EphA3 antibody has a CDR3 sequence GGYYEDFDS, SGYYEEFDS, SGVYEDFDS, SGYYEDFDV, or SGYYEDFDI; a V_{H} CDR1 that has the amino acid sequence GYWMN, TYWIS, or SYWIN; and a CDR2 that has the amino acid sequence DIYPGSGNTNYDEKFQG, DIYPGSGNTNYAQKFQG, DIYPGSGNTNYAQEFRG, DIYPGSGNTNYAQKFLG, DIYPGSGNTNYDEKFEG, or DIYPGSGNTNYDEKFKR. In some embodiments, the antibody has a V_{H} CDR3 sequence GGYYEDFDS, SGYYEEFDS, SGVYEDFDS, SGYYEDFDV, or SGYYEDFDI; a V_{H} CDR1 GYWMN; and a CDR2 DIYPGSGNTNYDEKFQG. In some embodiments, the antibody has a V_{H} CDR3 sequence GGYYEDFDS, SGYYEEFDS, SGVYEDFDS, SGYYEDFDV, or SGYYEDFDI; a V_{H} CDR1 TYWIS; and a V_{H} CDR2 DIYPGSGNTNYAQ(K/E)F(Q/R/L)G. In some embodiments, an antibody for use in the invention has the V_{H} CDR1, CDR2, and CDR3 from one of the V regions as shown in FIG. 4. In some embodiments, the antibody has a V_{H} V-segment amino acid sequence of a V-segment sequence shown in FIG. 4. In some embodiments, the V_{H} has the sequence of a VH region set forth in FIG. 4.

The invention also provides an anti-EphA3 antibody for use in the methods of the invention, e.g., for killing multiple myeloma cells, where the anti-EphA3 antibody has a V_{L} region that comprises a CDR3 binding specificity determinant having the sequence X₁X₂YX₃X₄YPYT, wherein X₁ is G, V, or A; X₂ is Q, R, or G; X₃ is A, S, or L; and X₄ is N or K. In some embodiments, the CDR3 comprises GQYANYPYT, VQYAKYPYT, AQYANYPYT, VQYSNYPYT, VQYANYPYT, VGYANYPYT, VRYANYPYT, or VQYLNYPYT. In some embodiments, the V_{L} region comprises a J segment that comprises at least 80% identity, typically at least 85% or 90% identity, to a human germline J segment amino acid sequence, or that differs no more than two amino acids from a human germline segment; and a V-segment that comprises at least 80% identity, typically at least 90% identity, or greater, to a human germ line V-segment amino acid sequence. In some embodiments, the J segment has no more than two amino acid changes, often no more than one amino acid change, relative to the sequence FGQGKLEIK from the human germ-line Jκ2 amino acid sequence and the V-segment comprises at least 90% identity to human germline JκI L15 amino acid sequence. In some embodiments, the FR4 of the antibody has the amino acid sequence FGQGKLEIK, or has no more than one amino acid residue changed relative to the sequence FGQGKLEIK. In some embodiments, the V_{L} region has a CDR3 GQYANYPYT, VQYAKYPYT, AQYANYPYT, VQYSNYPYT, VQYANYPYT, VGYANYPYT, VRYANYPYT, or VQYLNYPYT; and a CDR1 and CDR2 of a sequence V_{L} region shown in FIG. 4. For example, in some embodiments, the V_{L} region has a CDR3 GQYANYPYT, VQYAKYPYT, AQYANYPYT, VQYSNYPYT, VQYANYPYT, VGYANYPYT, VRYANYPYT, or VQYLNYPYT; a CDR1 sequence RASQGIISYLA, QASQDISTYLN, RASQEISGYLG, or RASQSISSYLA; and a CDR2 sequence AASSLQS, GASSLQS, AASSLQR, or AASTLDS. In some embodiments, the V_{L} region has a CDR3 GQYANYPYT, VQYAKYPYT, AQYANYPYT, VQYSNYPYT, VQYANYPYT, VGYANYPYT, VRYANYPYT, or VQYLNYPYT; a CDR1 RASQGIISYLA and a CDR2 GASSLQS. In some embodiments, the V_{L} region has a CDR3 GQYANYPYT, VQYAKYPYT, AQYANYPYT, VQYSNYPYT, VQYANYPYT, VGYANYPYT, VRYANYPYT, or VQYLNYPYT; a CDR1 QASQDISTYLN; and a CDR2 AASSLQR or AASSLQS. In some embodiments, the V_{L} region has a CDR3 GQYANYPYT, VQYAKYPYT, AQYANYPYT, VQYSNYPYT, VQYANYPYT, VGYANYPYT, VRYANYPYT, or VQYLNYPYT; a CDR1 sequence RASQSISSYLA and a CDR2 sequence AASSLQR. In some embodiments, the V_{L} region comprises the CDR1, CDR2, and CDR3 of one of the V_{L} regions set forth in FIG. 4. In some embodiments, the V_{L} region has a CDR3 as shown in FIG. 4 and comprises a V-segment that has a V-segment sequence as shown in FIG. 4. In some embodiments, the V_{L} region has the sequence of a V_{L} region set forth in FIG. 1.

In some embodiments, an anti-EphA3 antibody for use in the methods of the invention, e.g., for killing multiple myeloma cells, has a V_{H} region comprising a CDR3 having the sequence SGYYE(E/D)FDS and a V_{L} region CDR3 sequence set forth in the preceding paragraph. In some embodiments, the anti-EphA3 antibody comprises the V_{H} CDR1, CDR2, and CDR3 from one of the V_{H} regions set forth in FIG. 4 and the V_{L} CDR1, CDR2, and CDR3 from one of the V_{L} regions set forth in FIG. 4.

In some embodiments, ant anti-EphA3 antibody for use in the methods of the invention comprises a V_{H} region as set forth in FIG. 4 or a V_{L} region as set forth in FIG. 4. Often, the antibody comprises a V_{H} region as set forth in FIG. 4 and a V_{L} region as set forth in FIG. 4. In some embodiments, the antibody comprises one of the following combinations of V_{H} and V_{L} regions: a) SEQ ID NO:1 and SEQ ID NO:20, b) SEQ ID NO:2 and SEQ ID NO:11, c) SEQ ID NO: 2 and SEQ ID NO:12, d) SEQ ID NO:2 and SEQ ID NO:19, e) SEQ ID NO:2 and SEQ ID NO:21, f) SEQ ID NO:2 and SEQ ID NO:22, g) SEQ ID NO:2 and SEQ ID NO:23, h) SEQ ID NO:3 and SEQ ID NO:11, i) SEQ ID NO:3 and SEQ ID NO:12, j) SEQ ID NO:3 and SEQ ID NO:21, k) SEQ ID NO:3 and SEQ ID NO:22, l) SEQ ID NO:4 and SEQ ID NO:11, m) SEQ ID NO:4 and SEQ ID NO:13, n) SEQ ID NO:5 and SEQ ID NO:11, o) SEQ ID NO:5 and SEQ ID NO:13, p) SEQ ID NO:5 and SEQ ID NO:21, q) SEQ ID NO:6 and SEQ ID NO:14, r) SEQ ID NO:6 and SEQ ID NO:15, s) SEQ ID NO:7 and SEQ ID NO:14, t) SEQ ID NO:7 and SEQ ID NO:15, u) SEQ ID NO:8 and SEQ ID NO:14, v) SEQ ID NO:8 and SEQ ID NO:15, w) SEQ ID NO:9 and SEQ ID NO:16, x) SEQ ID NO:9 and SEQ ID NO:17, y) SEQ ID NO:9 and SEQ ID NO:19, z) SEQ ID NO:10 and SEQ ID NO:17, aa) SEQ ID NO:10 and SEQ ID NO:18, or bb) SEQ ID NO:10 and SEQ ID NO:20.

In some embodiments, an anti-EphA3 antibody for use in the invention has a V_{H} region sequence selected from the V_{H} region sequences in FIG. 1 and a V_{L} region selected from the V_{L} region sequences in FIG. 4, has a monovalent affinity better than about 10 nM, and often better than about 5 nM or 1 nM as determined by surface plasmon resonance analysis performed at 37° C. Thus, in some embodiments, the antibodies of the invention have an affinity (as measured using surface plasmon resonance) of about 10 nM, about 5 nM, about 2.5 nM, about 1 nM, about 0.5 nM, about 0.25 nM, or about 0.1 nM, or better.

In additional aspects, the invention provides a method of treating a patient suffering from multiple myeloma by administering an anti-EphA3 antibody as described herein. In some embodiments, the multiple myeloma patient is treated with additional chemotherapeutic agent or agents, which may be selected from the following agents or types of agents: melphalan, steroids (e.g., prednisone, dexamethasone), proteosome inhibitors (e.g., bortezomib), thalidomide, thalidomide derivatives (e.g., lenalidomide), dexamethasone, tyrosine kinase inhibitors (e.g., imatinib, dasitinib, or nilotinib), alkylating agents, antrhacyclines, Apo2L/TRAIL, HMG-CoA reductase inhibitors (e.g., lovastatin). Various combinations of the foregoing agents may be employed, in addition to administration of the antibody of the invention.

In a further aspect, the invention provides a method of identifying a multiple myeloma patient who is a candidate for treatment with an anti-EphA3 antibody as described herein, the method comprising detecting expression of EphA3 in multiple myeloma cells from the patient. In additional aspects of the invention, such a patient can be treated with an anti-EphA3 antibody as described herein. In some embodiments, the method comprises administering one or more additional therapeutic agents. In a further aspect, the invention provides a method of monitoring the efficacy of treatment of a patient having multiple myeloma, by measuring the expression of EphA3 in multiple myeloma cells derived from the patient.

In yet a further aspect, the invention provides a method of de-bulking multiple myeloma cells in a patient who is about to undergo a procedure for immunoablation for autologous or allogeneic hematopoietic stem cell transplantation. According to this aspect of the invention, anti-EphA3 antibodies of the invention are administered to the patient, prior to the procedure. In still another, related aspect, the patient's autologous stem cells may be treated with the anti-EphA3 antibodies, prior to reintroduction of the stem cells to the patient, in order to further reduce the number of tumor stem cells that are present in the transplanted hematopoietic stem cells.

### BRIEF DESCRIPTION OF THE DRAWINGS

FIG. 1 shows enhanced ADCC-induced cell killing of primary multiple myeloma cells incubated with increasing concentrations of a human engineered anti-EphA3 antibody of the invention.

FIG. 2 shows the results of studies in which various primary multiple myeloma cell samples were tested for binding of an anti-EphA3 antibody of the invention, cells were further characterized for the presence of certain other cell surface antigens (CD138, CD38, CD19, CD27), and antigens correlated with sensitivity to anti-EphA3 antibody mediated ADCC and apoptosis.

FIG. 3 provides data showing that Annexin V-staining apoptotic cells were identified by flow cytometry.

FIG. 4 provides examples of V_{H} and V_{L} sequences of anti-EphA3 antibodies for use in the invention.

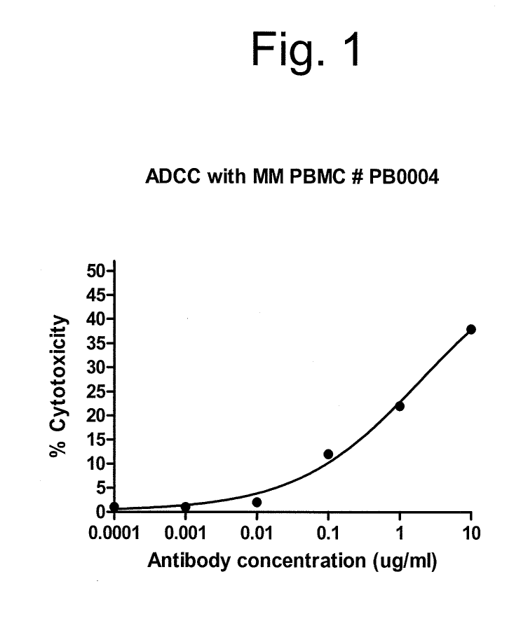

### DETAILED DESCRIPTION OF THE INVENTION

### Definitions

The term “multiple myeloma” as used herein refers to a disorder characterized by malignant proliferation of plasma cells derived from a single clone. It is diagnosed using standard diagnostic criteria. Typically, low red blood cell count and/or elevated protein levels in the blood or protein in the urine is an early indicator; a bone marrow biopsy showing high levels of myeloma cells (>10% plasma cells) in the bone marrow is more definitive. The presence of the M protein in the serum and/or presence of lytic lesions in the bones are also diagnostic indicators of the disorder.

The term “cancer cell” or “tumor cell” as used herein refers to a neoplastic cell. The term includes cancer cells that are benign as well as malignant. Neoplastic transformation is associated with phenotypic changes of the tumor cell relative to the cell type from which it is derived. The changes can include loss of contact inhibition, morphological changes, and aberrant growth. (see, Freshney, Culture of Animal Cells a Manual of Basic Technique (3^{rd} edition, 1994).

“Inhibiting growth of a cancer” in the context of the invention refers to slowing growth and/or reducing the tumor cell burden of a patient that has a multiple myeloma. “Inhibiting growth of a cancer” thus includes killing cancer cells as well as slowing or arresting cancer cell growth.

“Apoptosis” in the context of this invention is a programmed cell death by DNA degradation with specific endonuclease-DNA fragmentation. Loss of membrane phospholipid asymmetry and expression of phosphatidylserine on the outer membrane leaflet occur during apoptosis. Annexin V has a high affinity for phosphatidylserine. This characteristic may be used to detect apoptotic cell death by annexin V binding, e.g., using fluorescence-activated cell sorting (FACS).

Antibody-dependent cell-mediated cytotoxicity (ADCC) is an immune response in which antibodies, by coating target cells, makes the target cells vulnerable to attack by effector cells of the immune system.

As used herein “EphA3” refers to the Eph receptor A3. This receptor has also been referred to as “Human embryo kinase”, “hek”, “eph-like tyrosine kinase 1”, “etk1” or “tyro4”. EphA3 belongs to the ephrin receptor subfamily of the protein-tyrosine kinase family. EPH and EPH-related receptors have been implicated in mediating developmental events. Receptors in the EPH subfamily typically have a single kinase domain and an extracellular region containing a Cys-rich domain and 2 fibronectin type III repeats. The ephrin receptors are divided into 2 groups based on the similarity of their extracellular domain sequences and their affinities for binding ephrin-A and ephrin-B ligands. EphA3 binds ephrin-A ligands. EphA3 nucleic acid and protein sequences are known. An example of a human EphA3 amino acid sequence is available under accession number EAW68857.

For the purposes of the present invention, “activation” of EphA3 causes phosphorylation of EphA3 and apoptosis. An antibody that activates EphA3 or “an activating antibody” causes phosphorylation of EphA3 and apoptosis and is therefore considered to be an agonist in the context of this invention. EphA3 can be activated by dimerization, which leads to apoptosis. In some embodiments, an antibody that activates EphA3 competes with mAb IIIA4 for binding to EphA3. Typically, an “activating” antibody binds to the ligand binding domain (amino acids 29-202 of EphA3) wherein amino acid residues 131, 132, and 136 are important for binding. In some embodiments, the activating antibody binds to a site encompassing the residues 131, 132, and 136 within the ligand binding domain of human EphA3 protein.

In the present invention, “EphA3 antibody” or “anti-EphA3 antibody” are used interchangeably to refer to an antibody that specifically binds to EphA3. In some embodiments, the antibody can dimerize EphA3. The term encompasses antibodies that bind to EphA3 in the presence of ephrin ligand (e.g., ephrin-A5) binding, as well as antibodies that bind to the ligand binding site and block ligand binding.

An “EphA3 antibody that binds to EphA3 in the presence of binding of an ephrin ligand” refers to an antibody that does not inhibit binding of an ephrin ligand, such as ephrin-A5, to EphA3 when the antibody is present in a binding reaction comprising EphA3 and the ephrin ligand.

The term “mAb IIIA4” refers to monoclonal antibody IIIA4 that was originally raised against LK63 human acute pre-B leukemia cells to affinity isolate EphA3 (Boyd, et al. J Biol Chem 267:3262-3267, 1992). mAb IIIA4 binds to the native EphA3 globular ephrin-binding domain but does not prevent ligand binding (e.g., Smith, et al., J. Biol. Chem 279:9522-9531, 2004). The hybridoma is deposited in the European Collection of Animal Cell Cultures under accession no. 91061920 (see, e.g., EP patent no. EP0590030).

An “antibody having an active isotype” as used herein refers to an antibody that has a human Fc region that binds to an Fc receptor present on immune effector cells. “Active isotypes” include IgG1, IgG3, IgM, IgA, and IgE. The term encompasses antibodies that have a human Fc region that comprises modifications, such as mutations or changes to the sugar composition and/or level of glycosylation, that modulate Fc receptor binding.

An “Fc region” refers to the constant region of an antibody excluding the first constant region immunoglobulin domain. Thus, “Fc region” refers to the last two heavy chain constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 and Cγ3 and the hinge between Cγ1 and Cγ. It is understood in the art that the boundaries of the Fc region may vary, however, the human IgG heavy chain Fc region is usually defined to comprise residues C226 or P230 to its carboxyl-terminus, using the numbering is according to the EU index as in Kabat et al. (1991, NIH Publication 91-3242, National Technical Information Service, Springfield, Va.). The term “Fc region” may refer to this region in isolation or this region in the context of an antibody or antibody fragment. “Fc region” includes naturally occurring allelic variants of the Fc region as well as modifications that modulate effector function. Fc regions also include variants that don't result in alterations to biological function. For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, et al., Science 247:306-1310, 1990).

As used herein, an “antibody” refers to a protein functionally defined as a binding protein and structurally defined as comprising an amino acid sequence that is recognized by one of skill as being derived from the framework region of an immunoglobulin encoding gene of an animal producing antibodies. An antibody can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

The term “antibody” as used herein includes antibody fragments that retain binding specificity. For example, there are a number of well characterized antibody fragments. Thus, for example, pepsin digests an antibody C-terminal to the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined by digestion of an intact antibody, one of skill will appreciate that fragments can be synthesized de novo either chemically or by utilizing recombinant DNA methodology. Thus, the term “antibody”, as used herein, also includes antibody fragments either produced by the modification of whole antibodies or synthesized using recombinant DNA methodologies.

Antibodies include V_{H}-V_{L} dimers, including single chain antibodies (antibodies that exist as a single polypeptide chain), such as single chain Fv antibodies (sFv or scFv) in which a variable heavy and a variable light region are joined together (directly or through a peptide linker) to form a continuous polypeptide. The single chain Fv antibody is a covalently linked V_{H}-V_{L} which may be expressed from a nucleic acid including V_{H}- and V_{L}-encoding sequences either joined directly or joined by a peptide-encoding linker (e.g., Huston, et al. Proc. Nat. Acad. Sci. USA, 85:5879-5883, 1988). While the V_{H} and V_{L} are connected to each as a single polypeptide chain, the V_{H} and V_{L} domains associate non-covalently. Alternatively, the antibody can be another fragment. Other fragments can also be generated, e.g., using recombinant techniques, as soluble proteins or as fragments obtained from display methods. Antibodies can also include diantibodies and miniantibodies. Antibodies of the invention also include heavy chain dimers, such as antibodies from camelids. For the purposes of this inventor, antibodies are employed in a form that can activate EphA3 present on the surface of multiple myeloma cells or that can kill multiple myeloma cells by ADCC. Thus, in some embodiments an antibody is dimeric. In other embodiments, the antibody may be in a monomeric form that has an active isotype. In some embodiments the antibody is in a multivalent form, e.g., a trivalent or tetravalent form, that can cross-link EphA3.

As used herein, “V-region” refers to an antibody variable region domain comprising the segments of Framework 1, CDR1, Framework 2, CDR2, and Framework3, including CDR3 and Framework 4, which segments are added to the V-segment as a consequence of rearrangement of the heavy chain and light chain V-region genes during B-cell differentiation.

As used herein, “complementarity-determining region (CDR)” refers to the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol. 1997, 273(4)). Definitions of antigen combining sites are also described in the following: Ruiz et al., IMGT, the international ImMunoGeneTics database. Nucleic Acids Res., 28, 219-221 (2000); and Lefranc, M. P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl. Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

As used herein, “chimeric antibody” refers to an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region, or portion thereof, having a different or altered antigen specificity; or with corresponding sequences from another species or from another antibody class or subclass.

As used herein, “humanized antibody” refers to an immunoglobulin molecule in CDRs from a donor antibody are grafted onto human framework sequences. Humanized antibodies may also comprise residues of donor origin in the framework sequences. The humanized antibody can also comprise at least a portion of a human immunoglobulin constant region. Humanized antibodies may also comprise residues which are found neither in the recipient antibody nor in the imported CDR or framework sequences. Humanization can be performed using methods known in the art (e.g., Jones et al., Nature 321:522-525; 1986; Riechmann et al., Nature 332:323-327, 1988; Verhoeyen et al., Science 239:1534-1536, 1988); Presta, Curr. Op. Struct. Biol. 2:593-596, 1992; U.S. Pat. No. 4,816,567), including techniques such as “superhumanizing” antibodies (Tan et al., J. Immunol. 169: 1119, 2002) and “resurfacing” (e.g., Staelens et al., Mol. Immunol. 43: 1243, 2006; and Roguska et al., Proc. Natl. Acad. Sci. USA 91: 969, 1994).

A “HUMANEERED™” antibody in the context of this invention refers to an engineered human antibody having a binding specificity of a reference antibody. An engineered human antibody for use in this invention has an immunoglobulin molecule that contains minimal sequence derived from a donor immunoglobulin. In some embodiments, the engineered human antibody may retain only the minimal essential binding specificity determinant from the CDR3 regions of a reference antibody. Typically, an engineered human antibody is engineered by joining a DNA sequence encoding a binding specificity determinant (BSD) from the CDR3 region of the heavy chain of the reference antibody to human V_{H} segment sequence and a light chain CDR3 BSD from the reference antibody to a human V_{L} segment sequence. A “BSD” refers to a CDR3-FR4 region, or a portion of this region that mediates binding specificity. A binding specificity determinant therefore can be a CDR3-FR4, a CDR3, a minimal essential binding specificity determinant of a CDR3 (which refers to any region smaller than the CDR3 that confers binding specificity when present in the V region of an antibody), the D segment (with regard to a heavy chain region), or other regions of CDR3-FR4 that confer the binding specificity of a reference antibody. Methods for engineering human antibodies are provided in US patent application publication no. 20050255552 and US patent application publication no. 20060134098.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

The term “human antibody” as used herein refers to an antibody that is substantially human, i.e., has FR regions, and often CDR regions, from a human immune system. Accordingly, the term includes humanized and human engineered antibodies as well as antibodies isolated from mice reconstituted with a human immune system and antibodies isolated from display libraries.

A “hypofucosylated” antibody preparation refers to an antibody preparation in which the average content of α1,6-fucose is less than 50% of that found in naturally occurring IgG antibody preparations. As understood in the art, “hypofucosylated” is used in reference to a population of antibodies.

An “afucosylated” antibody lacks α1,6-fucose attached to the CH2 domain of the IgG heavy chain.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid. Similarly, a “heterologous” protein will often refer to two or more subsequences that are not found in the same relationship to each other in nature.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operable linkage of different sequences is achieved. Thus, an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The phrase “specifically (or selectively) binds” to an antibody or “specifically (or selectively) immunoreactive with,” when referring to a protein or peptide, refers to a binding an anti-EphA3 antibody typically binds to EphA3 with an affinity that is at least 100-fold better than its affinity for other antigens.

The term “equilibrium dissociation constant (K_{D}) refers to the dissociation rate constant (k_{d}, time⁻¹) divided by the association rate constant (kₐ, time₋₁, M⁻¹). Equilibrium dissociation constants can be measured using any known method in the art. The antibodies of the present invention are high affinity antibodies. Such antibodies have an affinity better than 500 nM, and often better than 50 nM or 10 nM. Thus, in some embodiments, the antibodies of the invention have an affinity in the range of 500 nM to 100 pM, or in the range of 50 or 25 nM to 100 pM, or in the range of 50 or 25 nM to 50 pM, or in the range of 50 nM or 25 nM to 1 pM.

As used herein, “cancer therapeutic agent” or “chemotherapeutic agent” refers to an agent that when administered to a patient suffering from cancer in a therapeutically effective dose, will cure or at least partially arrest the symptoms of the disease and complications associated with the disease.

The terms “identical” or percent “identity,” in the context of two or more polypeptide (or nucleic acid) sequences, refer to two or more sequences or subsequences that are the same or have a specified percentage of amino acid residues (or nucleotides) that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site). Such sequences are then said to be “substantially identical.” “Substantially identical” sequences also includes sequences that have deletions and/or additions, as well as those that have substitutions, as well as naturally occurring, e.g., polymorphic or allelic variants, and man-made variants. As described below, the preferred algorithms can account for gaps and the like. Preferably, protein sequence identity exists over a region that is at least about 25 amino acids in length, or more preferably over a region that is 50-100 amino acids=in length, or over the length of a protein.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

An indication that two polypeptides are substantially identical is that the first polypeptide is immunologically cross reactive with the antibodies raised against the second polypeptide. Thus, a polypeptide is typically substantially identical to a second polypeptide, e.g., where the two peptides differ only by conservative substitutions.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” in some embodiments denotes that a protein gives rise to essentially one band in an electrophoretic gel. Preferably, it means that the protein is at least 85% pure, more preferably at least 95% pure, and most preferably at least 99% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typical conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “a” or “an” is generally intended to mean “one or more” unless otherwise indicated.

### Introduction

The invention is based, in part, on the discovery that EphA3 is expressed on the surface of multiple myeloma cells in patients. In one aspect, the invention therefore relates to methods of killing multiple myeloma cells by contacting the cells with an anti-EphA3 antibody. Accordingly, the invention provides methods of treating multiple myeloma comprising administering an anti-EphA3 antibody to a patient.

In some embodiment, the anti-EphA3 antibody is an activating antibody. In some embodiments, an anti-EphA3 antibody for use in this invention does not block binding of EphA3 to ephrin, e.g., ephrin-A5. In some embodiments, the antibody dimerizes EphA3. In some embodiments, the antibody cross-links EphA3. In some embodiments, the antibody competes with Mab IIIA4 for binding to EphA3, e.g., such an antibody may bind to the same epitope as Mab IIIA4. In some embodiments, the antibody has an active isotype where the heavy chain constant domain can bind to Fc receptor present on immune effector cells, leading to ADCC.

### Anti-EphA3 Antibodies

Anti-EphA3 antibodies for use in the invention can be raised against EphA3 proteins, or fragments, or produced by in vitro methods, as outlined below using known methods. For example, Boyd et al., J. Biol. Chem. 267:3262-3287, 1992 describes a method for producing and characterizing monoclonal antibodies. Alternatively, anti-EphA3 antibodies are available commercially. Any number of techniques can be used to determine antibody binding specificity. See, e.g., Harlow & Lane, Antibodies, A Laboratory Manual (1988) for a description of immunoassay formats and conditions that can be used to determine specific immunoreactivity of an antibody.

In some embodiments, the anti-EphA3 antibody is a polyclonal antibody. Methods of preparing polyclonal antibodies are known to the skilled artisan (e.g., Harlow & Lane, Antibodies, A Laboratory manual (1988); Methods in Immunology). Polyclonal antibodies can be raised in a mammal by one or more injections of an immunizing agent and, if desired, an adjuvant. The immunizing agent includes a EphA3 receptor protein, or fragment thereof.

In some embodiments, the anti-EphA3 antibody is a monoclonal antibody. Monoclonal antibodies may be prepared using hybridoma methods, such as those described by Kohler & Milstein, Nature 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

Human monoclonal antibodies can be produced in vitro using various techniques known in the art, including phage display libraries (Hoogenboom & Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

In some embodiments the anti-EphA3 antibodies are chimeric or humanized monoclonal antibodies. As noted above, humanized forms of antibodies are chimeric immunoglobulins in which a CDR of a human antibody is replaced by a CDR of a non-human species such as mouse, rat or rabbit having the desired specificity, affinity and capacity.

An antibody that is employed in the methods of the invention can be in numerous formats. In some embodiments, the antibody can include an Fc region, e.g., a human Fc region. For example, such antibodies include IgG antibodies that bind EphA3 and that have an active isotype. In some embodiments, the antibody can be an active fragment (e.g., it can dimerize EphA3) or derivative of an antibody such as an Fab, Fab′, F(ab′)₂, Fv, scFv, or a single domain antibody (“dAb”). For example, in some embodiments, the antibody may be a F(ab′)₂. Other examples of embodiments of antibodies that can be employed in the invention include activating nanobodies or activating camellid antibodies. As noted above, such antibodies can be produced using recombinant techniques well known to persons of skill in the art. As appreciated by one of skill in the art, in some embodiments when an antibody is in a format that can be monovalent, e.g., an Fv or Fab format, the antibody may be employed as a multivalent antibody, such as a trivalent or tetravalent antibody. Methods of generating multivalent antibodies re known (see, e.g., King et al., Cancer Res. 54:6176-6185, 1994; WO02/096948).

In many embodiments, an antibody for use in the invention has an Fc constant region that has an effector function, e.g., binds to an Fc receptor present on immune effector cells. Exemplary “effector functions” include C1q binding; complement dependent cytotoxicity; Fc receptor binding; antibody-dependent cell-mediated cytotoxicity (ADCC); phagocytosis; down regulation of cell surface receptors (e.g., B cell receptor), and the like. Such effector functions generally require the Fc region to be combined with a binding domain (e.g. an antibody variable domain) and can be assessed using known assays (see, e.g., the references cited hereinbelow.)

Anti-EphA3 antibodies that have an active isotype and are bound to Fc-receptors on effector cells, such as macrophages, monocytes, neutrophils and NK cells, can induce cell death by ADCC.

The Fc region can be from a naturally occurring IgG1, or other active isotypes, including IgG3, IgM, IgA, and IgE. “Active isotypes” include antibodies where the Fc region comprises modifications to increase binding to the Fc receptor or otherwise improve the potency of the antibody. Such an Fc constant region may comprise modifications, such as mutations, changes to the level of glycosylation and the like, that increase binding to the Fc receptor. There are many methods of modifying Fc regions that are known in the art. For example, U.S. Patent Application Publication No. 20060039904 describes variants of Fc receptors that have enhanced effector function, including modified binding affinity to one or more Fc ligands (e.g., FcγR, C1q). Additionally, such Fc variants have altered antibody-dependent cell-mediated cytotoxicity (ADCC) and/or complement dependent cytotoxicity (CDC) activity. Other Fc variants include those disclosed by Ghetie et al., Nat. Biotech. 15:637-40, 1997; Duncan et al, Nature 332:563-564, 1988; Lund et al., J. Immunol. 147:2657-2662, 1991; Lund et al, Mol Immunol 29:53-59, 1992; Alegre et al, Transplantation 57:1537-1543, 1994; Hutchins et al., Proc Natl. Acad Sci USA 92:11980-11984, 1995; Jefferis et al, Immunol Lett. 44:111-117, 1995; Lund et al., FASEB J 9:115-119, 1995; Jefferis et al, Immunol Lett 54:101-104, 1996; Lund et al, J Immunol 157:4963-4969, 1996; Armour et al., Eur J Immunol 29:2613-2624, 1999; Idusogie et al, J Immunol 164:4178-4184, 200; Reddy et al, J Immunol 164:1925-1933, 2000; Xu et al., Cell Immunol 200:16-26, 2000; Idusogie et al, J Immunol 166:2571-2575, 2001; Shields et al., J Biol Chem 276:6591-6604, 2001; Jefferis et al, Immunol Lett 82:57-65. 2002; Presta et al., Biochem Soc Trans 30:487-490, 2002; Lazar et al., Proc. Natl. Acad. Sci. USA 103:4005-4010, 2006; U.S. Pat. Nos. 5,624,821; 5,885,573; 5,677,425; 6,165,745; 6,277,375; 5,869,046; 6,121,022; 5,624,821; 5,648,260; 6,194,551; 6,737,056; 6,821,505; 6,277,375; 7,335,742; and 7,317,091; and PCT Publications WO 94/2935; WO 99/58572; WO 00/42072; WO 02/060919, and WO 04/029207,

In some embodiments, the glycosylation of Fc regions may be modified for example, a modification may be aglycosylation, for example, by altering one or more sites of glycosylation within the antibody sequence. Such an approach is described in further detail in U.S. Pat. Nos. 5,714,350 and 6,350,861. An Fc region can also be made that has an altered type of glycosylation, such as a hypofucosylated Fc variant having reduced amounts of fucosyl residues or an Fc variant having increased bisecting GlcNAc structures. Such carbohydrate modifications can be accomplished by, for example, expressing the antibody in a host cell with altered glycosylation machinery. Cells with altered glycosylation machinery, including yeast and plants, have been described in the art and can be used as host cells in which to express recombinant antibodies of the invention to thereby produce an antibody with altered glycosylation. Techniques for modifying glycosylation include those disclosed e.g., in Umana et al, Nat. Biotechnol 17:176-180, 1999; Davies, et al., Biotechnol. Bioeng. 74:288-294, 2001; Shields et al, Biol Chem 277:26733-26740, 2002; Shinkawa et al., J Biol Chem 278:3466-3473, 2003; Niwa et al. Clinc. Cancer Res. 1-:6248-6255, 2004; Presta et al., Biochem Soc Trans 30:487-490, 2002; Kanda et al, Glycobiology 17:104-118, 2006; U.S. Pat. Nos. 6,602,684; 6,946,292; and 7,214,775; U.S. Patent Application Publication Nos. 20070248600; 20070178551; 20080060092; 20060253928; PCT publications WO 00/61739; WO 01/292246; WO 02/311140; and WO 02/30954; and POTELLIGENT® technology (Biowa, Inc. Princeton, N.J.); and GlycoMAb™. glycosylation engineering technology (GLYCART biotechnology AG, Zurich, Switzerland). In a hypofucosylated antibody preparation, typically at least 50 to 70% of the antibody molecule, often at least 80% of the molecules, or at least 90% of the molecules, lack fucose.

In some embodiments of the invention, the antibody is additionally engineered to reduce immunogenicity, e.g., so that the antibody is suitable for repeat administration. Methods for generating antibodies with reduced immunogenicity include humanization and humaneering procedures and modification techniques such as de-immunization, in which an antibody is further engineered, e.g., in one or more framework regions, to remove T cell epitopes.

In some embodiments, the antibody is a HUMANEERED™. A HUMANEERED™ antibody (also referred to herein as an engineered human antibody) is a human antibody that has been engineered to have a binding specificity of a reference antibody, obtained by joining a DNA sequence encoding a binding specificity determinant (BSD) from the CDR3 region of the heavy chain of the reference antibody to human V_{H} segment sequence and a light chain CDR3 BSD from the reference antibody to a human V_{L} segment sequence. Methods for generating such human engineered antibodies are provided in US patent application publication no. 20050255552 and US patent application publication no. 20060134098.

An antibody can further be de-immunized to remove one or more predicted T-cell epitopes from the V-region of an antibody. Such procedures are described, for example, in WO 00/34317.

In some embodiments, the variable region is comprised of human V-gene sequences. For example, a variable region sequence can have at least 80% identity, or at least 85% or at least 90% identity, to human germ-line V-gene sequences.

An antibody used in the invention can include a human constant region. The constant region of the light chain may be a human kappa or lambda constant region. The heavy chain constant region is often a gamma chain constant region, for example, a gamma-1 or gamma-3 constant region.

In some embodiments, e.g., where the antibody is a fragment, the antibody can be conjugated to another molecule, e.g., to provide an extended half-life in vivo such as a polyethylene glycol (pegylation) or serum albumin. Examples of PEGylation of antibody fragments are provided in Knight et al., Platelets 15:409, 2004 (for abciximab); Pedley et al., Br. J. Cancer 70:1126, 1994 (for an anti-CEA antibody); and Chapman et al., Nature Biotech. 17:780, 1999.

### Antibody Specificity

In some embodiments, an antibody for use in the invention activates EphA3 and/or kills EphA3⁺ cells by ADCC. An example of an antibody suitable for use with the present invention is an antibody that has the binding specificity of mAb IIIA4. The monoclonal antibody mAb IIIA4 binds to the native EphA3 globular ephrin-binding domain (Smith et al., J. Biol. Chem. 279:9522-9531, 2004; and Vearing et al., Cancer Res. 65:6745-6754, 2005). High affinity mAb IIIA4 binding to the EphA3 surface has little effect on the overall affinity of ephrin-A5 interactions with EphA3.

In some embodiments, a monoclonal antibody that competes with mAb IIIA4 for binding to EphA3, or that binds the same epitope as mAb IIIA4, is used. Any of a number of competitive binding assays can be used to measure competition between two antibodies for binding to the same antigen. For example, a sandwich ELISA assay can be used for this purpose. In an exemplary assay, ELISA is carried out by using a capture antibody to coat the surface of a well. A subsaturating concentration of tagged-antigen is then added to the capture surface. This protein will be bound to the antibody through a specific antibody:antigen interaction. After washing, a second antibody that is linked to a detectable moiety is added to the ELISA. If this antibody binds to the same site on the antigen as the capture antibody, or interferes with binding to that site, it will be unable to bind to the target protein as that site will no longer be available for binding. If however this second antibody recognizes a different site on the antigen it will be able to bind. Binding can be detected by quantifying the amount of detectable label that is bound. The background is defined by using a single antibody as both capture and detection antibody, whereas the maximal signal can be established by capturing with an antigen specific antibody and detecting with an antibody to the tag on the antigen. By using the background and maximal signals as references, antibodies can be assessed in a pair-wise manner to determine specificity. The ability of a particular antibody to recognize the same epitope as another antibody is typically determined by such competition assays.

A first antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays described above.

### Binding Affinity

In some embodiments, the antibodies suitable for use with the present invention have a high affinity binding for human EphA3. For the purposes of this invention, high affinity binding between an antibody and an antigen exists if the dissociation constant (K_{D}) of the antibody is <about 10 nM, for example, about 5 nM, or about 2 nM, or about 1 nM, or less. A variety of methods can be used to determine the binding affinity of an antibody for its target antigen such as surface plasmon resonance assays, saturation assays, or immunoassays such as ELISA or RIA, as are well known to persons of skill in the art. An exemplary method for determining binding affinity is by surface plasmon resonance analysis on a BIAcore™ 2000 instrument (Biacore AB, Freiburg, Germany) using CM5 sensor chips, as described by Krinner et al., (2007) Mol. Immunol. February; 44(5):916-25. (Epub 2006 May 11)).

The anti-EphA3 antibody can bind to any extracellular domain region of EphA3. In some embodiments, the anti-EphA3 antibody binds to the ephrin binding domain of EphA3. In some embodiments, the anti-EphA3 antibody activates EphA3. Often, the antibody dimerizes EphA3. In some embodiments, the antibody clusters EphA3. In some embodiments, an anti-EphA3 antibody can also be employed that has an active isotype, such as an IgG1, IgG3, IgM, IgA, or IgE, and is cytotoxic to multiple myeloma cells via ADCC. In other embodiments, the anti-EphA3 antibody has a non-active isotype, such as IgG2 or IgG4, in which case, without being bound by theory, its cytotoxic effects are thought to be predominantly attributable to apoptosis. Antibodies for use in the invention can also be multivalent including forms of monomers, such as Fab′ s, that are cross-linked or otherwise multimerized (Fab₂, Fab₄) to form multivalent antibodies.

In some embodiments, an antibody employed in the invention does not compete with an EphA3 ligand for binding to EphA3, whereas in other embodiments an EphA3 antibody for use in the invention can compete for binding of an EphA3 ligand such as an ephrin, e.g., ephrin-A5, to EphA3. Antibodies that compete with a ligand for binding to EphA3, can be identified using techniques as described above, where an ephrin ligand such as ephrin-A5, is used instead of another antibody for a competition analysis.

In exemplary embodiments, the anti-EphA3 antibody comprises the V_{L} and V_{H} regions of mAb IIIA4. In other embodiments, the anti-EphA3 antibody comprises CDRs 1, 2 and 3 of mAb IIIA4. In some embodiments, the anti-EphA3 antibody comprises CDR3 of mAb IIIA4. Table 1 provides CDR sequences (defined according to Kabat numbering) of antibodies that bind to the same epitope as mAb IIIA4. Affinity for EphA3 antigen was determined by ELISA. An antibody of the invention may thus also have heavy chain and/or lights chain CDRs set forth in Table 1.

TABLE 1

| | | | | AFFINITY |
|---|---|---|---|---|
| antibody | CDRH1 | CDRH2 | CDRH3 | (nM) |
| IIIA4 | SYWIN | DIYPGSGNTN | SGYYEDFDS | 2.5 |
| | | YDEKFKR | | |
| FA3AM-H12A | TYWIS | DIYPGSGNTN | SGYYEEFDS | 3.2 |
| | | YDEKFQG | | |
| K3D | TYWIS | DIYPGSGNTN | SGYYEEFDS | 25 |
| | | YDEKFEG | | |
| FA225-C10 | GYWMN | DIYPGSGNTN | GGYYEDFDS | 1.2 |
| | | YDEKFQG | | |
| | | | | AFFINITY |
| antibody | CDRL1 | CDRL2 | CDRL3 | (nM) |
| IIIA4 | RASQE | AASTLDS | VQYANYPYT | 2.5 |
| | ISGYLG | | | |
| FA3AM-H12A | RASQG | AASSLQS | VQYANYPYT | 3.2 |
| | IISYLA | | | |
| K3D | RASQG | AASSLQS | VQYMNYPYT | 25 |
| | IISYLA | | | |
| FA225-C10 | RASQG | AASSLQS | GQYANYPYT | 1.2 |
| | IISYLA | | | |

Antibodies as described herein for use in the invention can be identified using known assays for the characteristic of interest. Thus, antibodies can be identified by screening for the ability to activate EphA3 (e.g., using an apoptosis assay as described in the examples), the ability to induce ADCC (e.g., using an ADCC assay as described in the examples), and for binding specificity and affinity using assays described above.

### Non Antibody EphA3 Binding Agents

Other proteins that bind to EphA3 and dimerize or activate EphA3 receptor may also be administered to a patient that has a leukemia or CMPS. Such proteins include a soluble Ephrin A5-Fc protein.

Other EphA3 binding agents include scaffolded proteins that bind EphA3. Thus, the EphA3 binding agent can be an “antibody mimetic” that targets and binds to the antigen in a manner similar to antibodies. When an antibody mimetic is used, the form of the mimetic is such that it dimerizes EphA3. For example, the antibody mimetic may be used in a dimeric or multivalent format.

Certain antibody mimetics use non-immunoglobulin protein scaffolds as alternative protein frameworks for the variable regions of antibodies. For example, Ku et al. (Proc. Natl. Acad. Sci. U.S.A. 92:6552-6556, 1995) discloses an alternative to antibodies based on cytochrome b562 in which two of the loops of cytochrome b562 were randomized and selected for binding against bovine serum albumin. The individual mutants were found to bind selectively with BSA similarly with anti-BSA antibodies.

U.S. Pat. Nos. 6,818,418 and 7,115,396 disclose an antibody mimic featuring a fibronectin or fibronectin-like protein scaffold and at least one variable loop. Known as Adnectins, these fibronectin-based antibody mimics exhibit many of the same characteristics of natural or engineered antibodies, including high affinity and specificity for any targeted ligand. The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions which would normally break down antibodies. In addition, since the structure of these fibronectin-based antibody mimics is similar to that of the IgG heavy chain, the process for loop randomization and shuffling may be employed in vitro that is similar to the process of affinity maturation of antibodies in vivo.

Beste et al. (Proc. Natl. Acad. Sci. U.S.A. 96:1898-1903, 1999) disclose an antibody mimic based on a lipocalin scaffold (Anticalin®). Lipocalins are composed of a β-barrel with four hypervariable loops at the terminus of the protein. The loops were subjected to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin® would be suitable to be used as an alternative to antibodies. Thus, Anticalins® are small, single chain peptides, typically between 160 and 180 residues, which provides several advantages over antibodies, including decreased cost of production, increased stability in storage and decreased immunological reaction.

U.S. Pat. No. 5,770,380 discloses a synthetic antibody mimetic using the rigid, non-peptide organic scaffold of calixarene, attached with multiple variable peptide loops used as binding sites. The peptide loops all project from the same side geometrically from the calixarene, with respect to each other. Because of this geometric confirmation, all of the loops are available for binding, increasing the binding affinity to a ligand. However, in comparison to other antibody mimics, the calixarene-based antibody mimic does not consist exclusively of a peptide, and therefore it is less vulnerable to attack by protease enzymes. Neither does the scaffold consist purely of a peptide, DNA or RNA, meaning this antibody mimic is relatively stable in extreme environmental conditions and has a long life span. Further, since the calixarene-based antibody mimic is relatively small, it is less likely to produce an immunogenic response.

Murali et al. (Cell Mol Biol 49:209-216, 2003) describe a methodology for reducing antibodies into smaller peptidomimetics, they term “antibody like binding peptidomimetics” (ABiP) which may also be useful as an alternative to antibodies.

WO 00/60070 discloses a polypeptide chain having CTL4A-like β-sandwich architecture. The peptide scaffold has from 6 to 9 β-strands, wherein two or more of the polypeptide β-loops constitute binding domains for other molecules, such as antigen binding fragments. The basic design of the scaffold is of human origin, thus reducing the risk of inducing an immune response. The O-sandwich scaffold may have improved stability and pharmacokinetic properties in vivo when compared to standard antibodies as the molecule contains a second, non-immunoglobulin disulphide bridge. As antigen binding domains can be located at opposite ends of a single peptide chain, the β-sandwich also facilitates design of bispecific monomeric molecules.

In addition to non-immunoglobulin protein frameworks, antibody properties have also been mimicked in compounds comprising RNA molecules and unnatural oligomers (e.g., protease inhibitors, benzodiazepines, purine derivatives and beta-turn mimics). Accordingly, non-antibody EphA3 binding agents can also include such compounds.

In some embodiments, the EphA3 binding agents employed in the invention competed with mAb IIIA4 for binding to EphA3. Such agents can be identified using known assays, such as the exemplary competition assays described herein.

Identification of Patients Who are Candidates for Treatment with Anti-EphA3

The invention also provides methods of determining whether a patient having a multiple myeloma is a candidate for treatment with an anti-EphA3 antibody. The methods comprise detecting the expression of EphA3 on multiple myeloma cells from the patient. In some embodiments, expression of EphA3 is detected on malignant plasma cells. In some embodiments, EphA3 expression is detected on the multiple myeloma tumor stem cells. In some embodiments, EphA3 expression is detected on both the malignant plasma cells and on the tumor stem cells.

EphA3 expression can be detected using methods well known in the art. Often, an immunological assay can be used to detect levels of EphA3 protein. Immunological assays include ELISA, immunocytochemistry, immunohistochemistry, fluorescent-activated cell sorting, and the like. In some embodiments, an antibody as described herein is used to detect EphA3 expression. Alternatively, EphA3 expression can be detected by detecting the level of mRNA encoding EphA3. Often, a nucleic acid amplification method, e.g., an RT-PCR is employed to quantify the amount of RNA.

A sample comprising multiple myeloma cells is obtained from the patient for evaluating EphA3 expression. The sample is often isolated from a bone marrow aspirate and peripheral blood, but other suitable samples may also be analyzed.

A patient is considered to be a candidate for treatment with an anti-EphA3 antibody if the multiple myeloma cells isolated from the patient express EphA3. Accordingly, “an EphA3⁺ patient” as used here is a patient that shows EphA3 expression on multiple myeloma cells. As demonstrated below, the presence or absence of other antigenic markers, such as CD138, CD19, CD27, may also be predictive of treatment candidacy, specifically, CD138⁻, CD19⁺ and CD27⁺ may all be indicative of the presence of EphA3 on the tumor stem cells in multiple myeloma.

The surface phenotype of alternative myelomagenic stem cell types is described in Ghosh and Matsui (Cancer Lett. 22009). The anti-EphA3 antibody may be used to treat myeloma patients whose tumor contains cells with a memory B-cell phenotype (CD19+, CD27+) or a plasma cell phenotype (CD38+, CD138+, CD45⁻) tumor stem cell population.

The presence or absence of antigenic markers can be determined using well known techniques, e.g., flow cytometry. Cells are considered to be positive for an antigenic marker when the level of the protein on the cell surface is above background.

### Treatment of Multiple Myeloma

In one aspect, the methods of the present invention comprise administering an anti-EphA3 agent, typically an anti-EphA3 antibody, to a patient that has multiple myeloma. Patients treated in accordance with the invention have multiple myeloma tumor cells that express EphA3. In some embodiments, an anti-EphA3 agent, such as an antibody, is administered to a patient that has a multiple myeloma tumor that has cells that express EphA3 and are characterized as: CD138⁻ and CD19⁺; CD19⁺, CD27⁺; or as CD38⁺, CD138⁺, CD45⁻.

Multiple myeloma stem cells can be identified by commonly used techniques such as immunophenotyping using flow cytometry, or by in vitro cell culture in lymphocyte conditioned media and serial replating in methylcellulose media r in vivo transplantation experiments.

In vivo transplantation to identify stem cells is carried out by passaging by serial transfer in mice with defective immune systems (SCID/NOD mice; van Rhenen et al., Clin. Cancer. Res. 11: 6520-6527, 2005).

The anti-EphA3 composition can be formulated for use in a variety of drug delivery systems. One or more physiologically acceptable excipients or carriers can also be included in the compositions for proper formulation. Suitable formulations for use in the present invention are found in Remington: The Science and Practice of Pharmacy, 21st Edition, Philadelphia, Pa. Lippincott Williams & Wilkins, 2005. For a brief review of methods for drug delivery, see, Langer, Science s249: 1527-1533 (1990).

The anti-EphA3 antibody for use in the methods of the invention is provided in a solution suitable for injection into the patient such as a sterile isotonic aqueous solution for injection. The anti-EphA3 antibody is dissolved or suspended at a suitable concentration in an acceptable carrier. In some embodiments the carrier is aqueous, e.g., water, saline, phosphate buffered saline, and the like. The compositions may contain auxiliary pharmaceutical substances as required to approximate physiological conditions, such as pH adjusting and buffering agents, tonicity adjusting agents, and the like.

The pharmaceutical compositions of the invention are administered to a patient that has a multiple myeloma in an amount sufficient to at least partially arrest the disease or symptoms of the disease and its complications. An amount adequate to accomplish this is defined as a “therapeutically effective dose.” A therapeutically effective dose is determined by monitoring a patient's response to therapy. Typical benchmarks indicative of a therapeutically effective dose are known in the art, depending on the disease. For example, therapeutic efficacy may be indicated by the decrease of the number of abnormal myeloid cells that are characteristic of the particular myeloid proliferation disorder in the blood or bone marrow.

The dose of the anti-EphA3 antibody is chosen in order to provide effective therapy for the patient and is in the range of about 0.1 mg/kg body weight to about 25 mg/kg body weight or in the range about 1 mg to about 2 g per patient. The dose is often in the range of about 0.5 mg/kg or about 1 mg/kg to about 10 mg/kg, or approximately about 50 mg to about 1000 mg/patient. In some embodiments, the antibody is administered in an amount less than about 0.1 mg/kg body weight, e.g., in an amount of about 20 mg/patient or less. The dose may be repeated at an appropriate frequency which may be in the range once per day to once every three months, depending on the pharmacokinetics of the antibody (e.g. half-life of the antibody in the circulation) and the pharmacodynamic response (e.g. the duration of the therapeutic effect of the antibody). In some embodiments where the antibody or modified antibody fragment has an in vivo half-life of between about 7 and about 25 days and antibody dosing is repeated between once per week and once every 3 months. In other embodiments, the antibody is administered approximately once per month.

Amounts that are administered that are effective will depend upon the severity of the disease and the general state of the patient's health, including other factors such as age, weight, gender, administration route, etc. Single or multiple administrations of the anti EphA3 antibody may be administered depending on the dosage and frequency as required and tolerated by the patient. In any event, the methods provide a sufficient quantity of the anti EphA3 antibody to effectively treat the multiple myeloma, alone or in conjunction with other chemotherapeutic agents or ablative therapies.

An anti-EphA3 antibody or anti-EphA3 agonist binding agent, e.g., that induces dimerization or activates EphA3, can be used in combination with one or more additional therapeutic agents to treat the multiple myeloma. Therapeutic agents that can be administered in conjunction with anti-EphA3 binding agents include agents or types of agents: melphalan, steroids (e.g., prednisone, dexamethasone), proteosome inhibitors (e.g., bortezomib), thalidomide, thalidomide derivatives (e.g., lenalidomide), dexamethasone, tyrosine kinase inhibitors (e.g., imatinib, dasitinib, nilotinib), alkylating agents, anthracyclines, Apo2L/TRAIL, HMG-CoA reductase inhibitors (e.g., lovastatin).

Specific regimens that can be used in conjunction with anti-EphA3 therapy include combinations of any of the above, and by way of example, but not limitation: melphalan and prednisone, melphalan and prednisone in conjunction with bortezomib; lenalidomide or thalidomide in conjunction with low dose dexamethasone, dasitinib, nilotinib, or imatinib.

In some embodiments an anti-EphA3 antibody or anti-EphA3 agonist binding agent, e.g., that induces dimerization or activates EphA3 can be used in combination with one or more additional therapeutic agents to treat a patient that has multiple myeloma where the multiple myeloma cells from the patient express EphA3.

Patients can receive one or more of these additional therapeutic agents as concomitant therapy. Alternatively, patients may be treated sequentially with additional therapeutic agents.

Anti-EphA3 antibody therapy may also be used in conjunction with or following immunoablative chemotherapy followed by autologous or allogeneic hematopoietic stem cell transplantation. Anti-EphA3 antibody therapy may also be used as a “de-bulking” treatment, in advance of such transplantation. Anti-EphA3 antibodies of the invention may further be used in the process of treating a patient's hematopoietic stem cells in vitro, prior to re-introducing such cells into the patient. This step would have the advantage of killing any residual tumor cells and thereby reducing the likelihood of recurrence of the disease.

In some embodiments, an anti-EphA3 antibody, or other activating Epha3 binding agent, is administered by injection or infusion through any suitable route including but not limited to intravenous, subcutaneous, intramuscular, intranasal, or intraperitoneal routes. In some embodiments, the anti EphA3 antibody is diluted in a physiological saline solution for injection prior to administration to the patient. The antibody is administered, for example, by intravenous infusion over a period of between 15 minutes and 2 hours.

The following examples are provided by way of illustration only and not by way of limitation. Those of skill in the art will readily recognize a variety of non-critical parameters that could be changed or modified to yield essentially similar results.

### EXAMPLES

### Example 1

### Identification of Multiple Myeloma Cells that Express EphaA3

Flow cytometry was used to evaluate the expression of EphA3 on the surface of cells from patients diagnosed with multiple myeloma. Multiple myeloma mononuclear cells from the peripheral blood and bone marrow aspirates were prepared by Ficoll-paque density gradient centrifugation. Cells isolated form peripheral blood (buffy coat cell preparations) or bone marrow samples were suspended at 1×10⁶ cells/0.1 ml in flow cytometry buffer (PBS, 2 mM EDTA, 2% fetal bovine serum, 0.05% sodium azide) with 1 μg normal IgG to block Fc-receptor binding (rat IgG; US Biological or anti-FcR antibodies). Anti-EphA3 antibody or negative control human IgG1 was added at 5 μg/ml and incubated on ice for 20 min. Cells were washed by dilution in flow cytometry buffer and centrifugation at 1000 rpm for 5 min. The cell pellet was resuspended in FITC-conjugated goat F(ab)′₂ anti-human IgG antibody (Caltag) diluted in flow cytometry buffer (1:20) and incubated on ice for 20 min. Cells were washed once by centrifugation and resuspended in flow cytometry buffer containing propidium iodide (Sigma) diluted 1:1000. Viable cells which exclude propidium iodide were analyzed by flow cytometry to identify EphA3-expressing cells in comparison with cells stained with negative control antibody.

Multiple myeloma stem cells may be characterized by the absence of CD138 antigen and presence of CD19 antigen and/or CD27 antigen (CD138⁻CD19⁺; CD138⁻CD27⁺; CD138⁻CD27⁺CD19⁺), by staining with antibodies to CD138, CD19 and CD27. EphA3 was detectable on the cell surface in several (3 out of 5) multiple myeloma patients tested; in these patients, the prevalence of EphA3+ cells ranged from 0-90% of cells (summarized in Table 2, below)

### Example 2

### Evaluation of the Ability of an Antibody to Induce Apoptosis

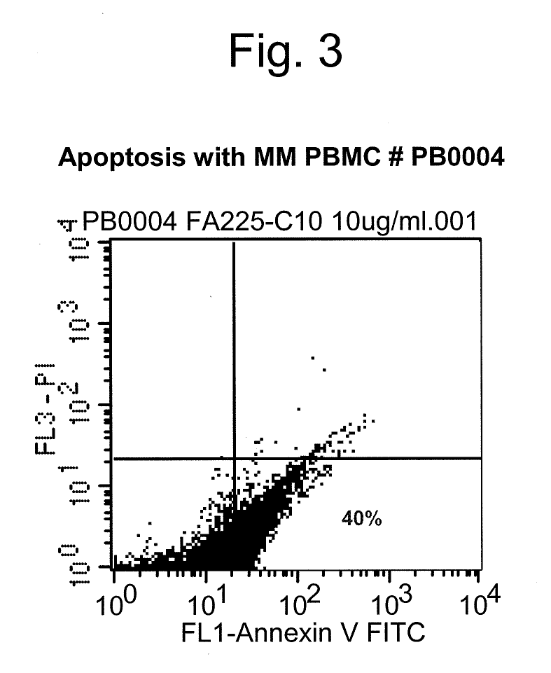

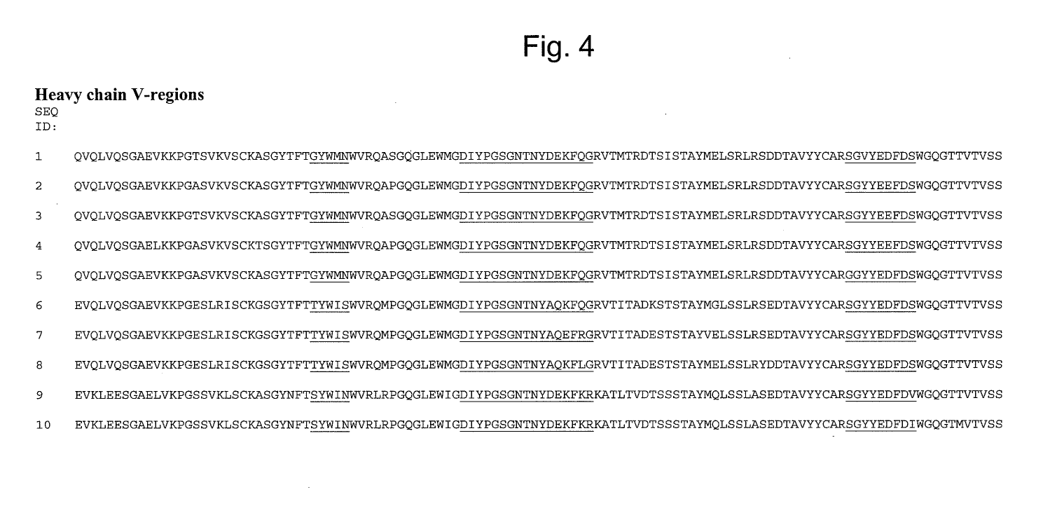

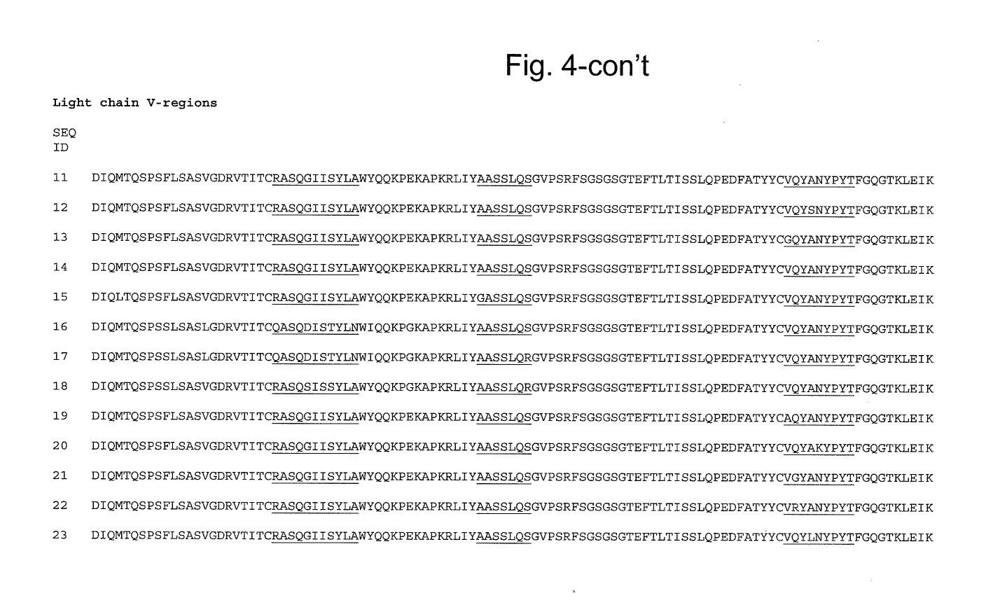

An engineered human activating antibody that binds to EphA3 was evaluated for the ability to induce apoptosis in vitro in primary cells isolated from patients or individuals suffering from multiple myeloma. Cells were seeded at 2×10⁵ cells/well in 96-well “U”-bottom plates in 0.1 ml culture medium (RPMI 1640 with 10% fetal bovine serum). Anti-EphA3 antibody or human IgG1 isotype control antibody was added to final concentrations between 10 μg/ml and 1 ng/ml and the plates were incubated at 37° C. and 5% carbon dioxide in a tissue-culture incubator for 24 hours. As a positive control for apoptosis induction, separate cell samples were incubated with camptothecin (10 μM; Calbiochem). At the end of the incubation, cells were harvested in to 5 ml culture tube and washed by centrifugation at 1000 rpm for 5 min followed by incubation in 0.1 ml buffer containing 5 μl FITC-conjugated Annexin V (BD Pharmingen) and 5 μl propidium iodide (BD Pharmingen) for 15 minutes at room temperature in the dark. Add 400 μl of 1× binding buffer to each tube and Annexin V-staining apoptotic cells were identified by flow cytometry (FIG. 3).

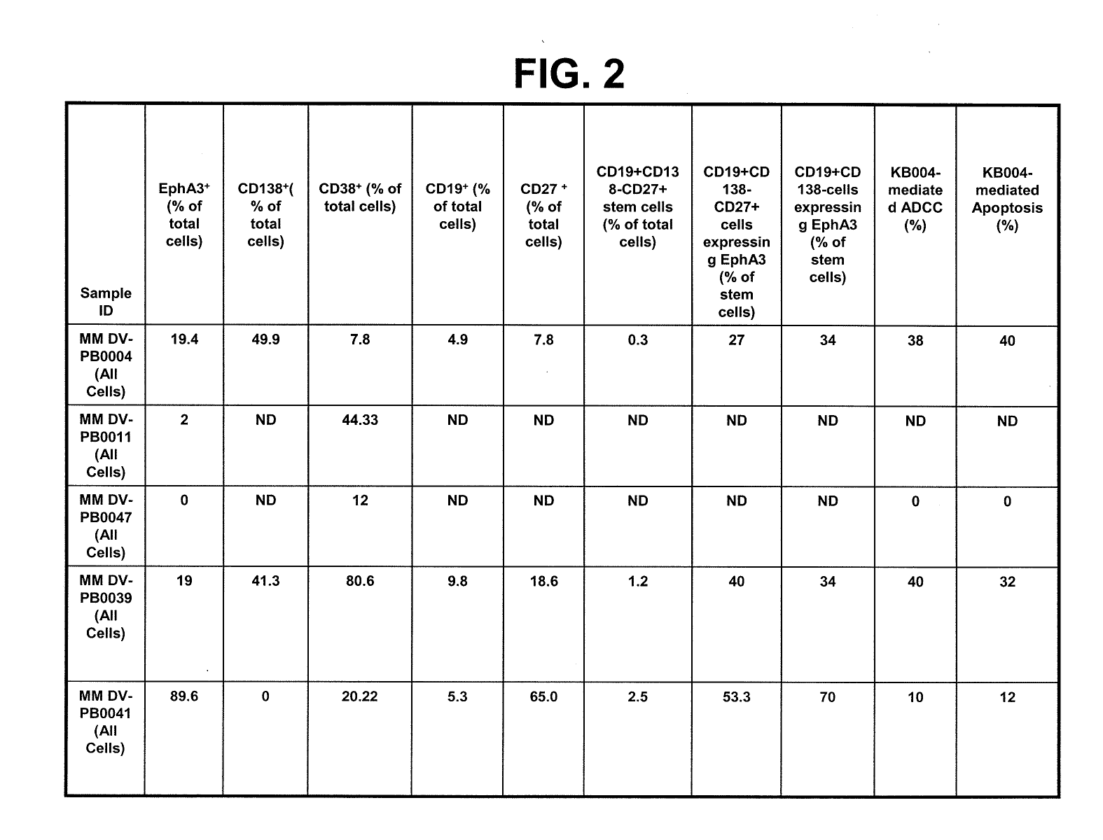

The results shown in FIG. 2 demonstrate that an anti-EphA3 antibody of the invention induced detectable levels of ADCC and apoptosis in cell samples from patients that exhibited more than about 19% EphA3⁺ multiple myeloma cells.

### Example 3

### Evaluation of ADCC Activity

Preparation of Anti-EphA3 Antibody Deficient in α1,6-fucose

To generate hypofucosylated antibody, a human engineered anti-EphA3 antibody (IgG1 κ) was expressed in a POTELLIGENT® CHOK1SV cell line (Biowa Inc., Princeton, N.J. and Lonza Ltd, Basel, Switzerland). This cell line is deficient in α-1,6-fucosyl transferase due to homozygous disruption of the FUT8 gene. Consequently, antibody expressed from this cell line lacks fucose in the carbohydrate structure. Cells expressing recombinant engineered anti-EphA3 antibody (IgG1k) were cultured in CHO-SFM II medium (Invitrogen) containing 2 μg/ml kifunensine to generate antibody with a modified glycosylation pattern defective in α 1,6-fucose as described (Zhou et al., Biotechnol. Bioeng. 99:652-665, 2008). Antibody purified by Protein A affinity chromatography showed significant reduction in the level of α 1,6-fucose determined by binding of Lens culinaris Lectin (Sigma) on protein blots with less than 10% antibody molecules containing this sugar moiety.

### ADCC Assay

Human PBMC effector cells were isolated from buffy coat samples by Ficoll-hypaque density separation according to standard techniques. Primary mononuclear cells from bone marrow or peripheral blood from patients with multiple myeloma were used as target cells in ADCC assays. Tumor target cells were incubated for 16 hours with human effector cells at an effector:target ratio of 100:1 or 200:1 for PBMC. Lactate dehydrogenase (LDH) released from dead cells was determined by CytoTox 96 assay (Promega). In this assay, incubation of target cells with antibody in the absence of effector cells showed no detectable cytotoxicity.

The antibody preparation deficient in α 1,6 fucose was evaluated in comparison with fucosylated antibody in ADCC assays. A pre-B cell leukemia derived cell line LK63 was used as the target. The antibody deficient in α 1,6 fucose is significantly more potent than the fucosylated antibody in this assay. ADCC activity is detected with low levels of defucosylated antibody (0.1 ng/ml), a concentration at which fucosylated antibody shows no detectable ADCC activity.

The engineered anti-EphA3 antibody also showed potent ADCC activity against primary human multiple myeloma cells from peripheral blood from multiple myeloma patients as shown in FIG. 2, as described above. In general, EphaA3 expression by multiple myeloma cells correlated with sensitivity to killing by an anti-EphA3 antibody of the invention.

All publications, patent applications, accession numbers, and other references cited in this specification are herein incorporated by reference as if each individual publication or patent application were specifically and individually indicated to be incorporated by reference.

1. A method of treating a patient suffering from multiple myeloma, the method comprising administering an anti EphA3 antibody to the patient.

2. The method of claim 1, wherein the patient's multiple myeloma cells comprise cells that express EphA3.

3. The method of claim 1, wherein the multiple myeloma cells are characterized by lack of CD138 and presence of CD19 (CD138⁻CD19⁺).

4. The method of claim 1, wherein the multiple myeloma cells are characterized by lack of CD138 and presence of CD27 (CD138⁻CD27⁺).

5. The method of claim 1, wherein the multiple myeloma cells are characterized by lack of CD138 and presence of CD19 and CD27 (CD138⁻CD19⁺CD27⁺).

6. The method of claim 1, wherein the multiple myeloma cells are characterized as CD19⁺, CD27⁺; or as CD38⁺, CD138⁺, CD45⁻.

7. The method of claim 1, wherein the antibody activates EphA3.

8. The method of claim 1, wherein the antibody has ADCC activity.

9. The method of claim 1, wherein the antibody is hypofucosylated.

10. The method of claim 1, wherein the antibody dimerizes EphA3.

11-12. (canceled)

13. The method of claim 1, wherein the anti EphA3 antibody clusters EphA3.

14. The method of claim 1, wherein the anti EphA3 antibody competes for EphA3 binding with mAb IIIA4.

15. The method of claim 1, wherein the anti EphA3 antibody is a recombinant human antibody.

16-20. (canceled)

21. The method of claim 1, wherein the anti EphA3 antibody comprises the V_{H} and V_{L} region CDR1, CDR2 and CDR3 of mAb IIIA4.

22-27. (canceled)

28. The method of claim 1, further comprising administering at least one additional chemotherapeutic agent to the patient.

29-31. (canceled)

32. A method of determining that a patient is a candidate for treatment with an anti-EphA3 antibody, the method comprising:
providing a sample from the patient, where the sample comprises multiple myeloma disorder cells; and
detecting expression of EphA3.

33. The method of claim 32, wherein the step of detecting expression of EphA3 comprises detecting protein expression.

34-35. (canceled)

36. The method of claim 32, wherein the step of detecting expression of EphA3 comprises detecting EphA3 RNA levels.

37-38. (canceled)

39. A method of monitoring the efficacy of treatment of a patient having a multiple myeloma, the method comprising:
obtaining a sample comprising multiple myeloma cells from the patient following a therapeutic treatment for the multiple myeloma; and
detecting expression of EphA3 by malignant plasma cells and/or stem cells.
